(19) United States
(12) Patent Application Publication
(10) Pub. No.: US 2017/0216444 A1
(43) Pub. Date: Aug. 3, 2017

(54) PREPARATION FOR FORMING EMBOLI AND MICROCATHETER

(71) Applicant: KAGOSHIMA UNIVERSITY, Kagoshima-shi, Kagoshima (JP)

(72) Inventors: Tsutomu TAMAI, Kagoshima-shi, Kagoshima (JP); Takayuki TAKEI, Kagoshima-shi, Kagoshima (JP); Akio IDO, Kagoshima-shi, Kagoshima (JP); Masahiro YOSHIDA, Kagoshima-shi, Kagoshima (JP); Shinji SAKAI, Suita-shi, Osaka (JP)

(21) Appl. No.: 15/514,886

(22) PCT Filed: Oct. 19, 2015
(86) PCT No.: PCT/JP2015/079405

§ 371 Date: Mar. 28, 2017

(30) Foreign Application Priority Data
Oct. 20, 2014 (JP) 2014-213293

(51) Int. Cl. A61K 47/42; A61K 47/36; A61K 47/32; A61K 31/555; A61K 47/08; A61K 38/54; A61K 38/44; A61K 47/38; A61K 47/30

(52) U.S. Cl. CPC A61K 47/42; A61K 47/38; A61K 47/36; A61K 47/32; A61K 47/30; A61K 47/08; A61K 38/54; A61K 38/443; A61K 38/44; A61K 31/555

(57) ABSTRACT

An object is to provide a preparation for forming emboli highly safe in a living body and capable of retaining and controlled-releasing an anticancer agent, occluding a blood vessel when injected into the blood vessel, unlikely to be washed out and having a controlled decomposition time (i.e., occludes a blood vessel for a while and quickly decomposes to prevent the necrosis of the entire tissues when the function is completed). The preparation for forming emboli according to the present invention comprises a solution comprising a phenolic hydroxyl group-modified polymer represented by the following formula (1): wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group, a solution comprising at least one selected from a peroxidase, a laccase, a tyrosinase, a catalase and an iron porphyrin complex and a solution comprising hydrogen peroxide.

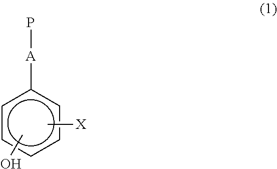

### TECHNICAL FIELD

The present invention relates to a preparation for forming emboli and a microcatheter.

### BACKGROUND ART

Transcatheter arterial embolization (TAE) has been conventionally known as a useful treatment method for regressing or eliminating cancer tissues such as a liver cancer wherein a gelatin sponge, porous gelatin particles or other spherical embolic substances are injected using a catheter into a blood vessel such as the hepatic artery delivering nutrition to cancer tissues so that the cancer tissues become ischemically necrotic by the occlusion of the blood vessel. Transcatheter arterial chemoembolization (TACE), utilizing the lodging of an oily contrast agent in a tumor, has been practiced wherein an embolic substance is injected after a mixed liquid (Lipiodol emulsion) of Lipiodol (registered trademark, iodized poppy seed oil fatty acid ethyl ester) and an anticancer agent is injected. The injection of the Lipiodol emulsion into the hepatic artery enables an anticancer agent to be released from the emulsion lodged in a tumor and thus the Lipiodol emulsion plays a role of drug delivery. A porous gelatin particle (Gelpart (registered trademark)) made from a pig collagen is known as such an embolic substance used in TACE.

Further, (Patent Literature 1) discloses as a preparation for forming emboli a composition comprising (a) about 2.5 wt % to about 8.0 wt % of a biocompatible polymer, (b) about 10 wt % to about 40 wt % of a water-insoluble biocompatible contrast agent having an average particle size of about 10 μm or less and (c) about 52 wt % to about 87.5 wt % of a biocompatible solvent selected from the group consisting of dimethyl sulfoxide, ethanol and acetone. Furthermore, (Patent Literature 2) discloses a method for temporarily forming an embolus at a vascular site of a mammal which includes a step for introducing a composition comprising a reverse thermosensitive polymer into an angioarchitecture of the mammal wherein the reverse thermosensitive polymer is gelled in the angioarchitecture and consequently an embolus is temporarily formed at the vascular site of the mammal.

Additionally, a microcatheter having a plural number of tubes (lumens) is known for sending a liquid to the circulatory system or for removing a liquid from the circulatory system of a mammal Such a microcatheter is used, for the purpose of treating a disease, to supply different drugs to the circulatory system of a patient or to send and remove blood using a plural number of lumens in dialysis. The microcatheter can also be used for injecting the embolic substance described above.

(Patent Literature 3) discloses as the microcatheter having a plural number of lumens, for example, a multilumen catheter to be inserted into the body of a mammal, the multilumen catheter provided with an elongated catheter body having a proximal end and a distal end and an outer wall surrounding at least three separated lumens and the lumens being the at least three separated lumens provided with the first, second and third lumens wherein the first lumen is disposed in the center relative to the second and third lumens and each of the first, second and third lumens has a distal opening positioned at a distal part of the catheter, wherein the distal openings of the second and third lumens are coadjacently disposed at an approximately same position along the longitudinal direction of the catheter and the distal opening of the first lumen is disposed distally or proximally relative to the distal openings of the second and third lumens along the longitudinal direction of the catheter.

Further, (Patent Literature 4) discloses a double lumen catheter having a catheter body of a tube with a blood returning lumen and a blood removing lumen partitioned with a partition wall, wherein a blood returning hole, i.e., the opening of the blood returning lumen, is arranged close to the tip of the catheter body and a blood removing hole, i.e., the opening of the blood removing lumen, is arranged at a position separated 3 to 11 cm from the tip of the catheter body toward the base side, wherein an opening surface of the blood removing hole has an angle of 5 to 90° relative to the longitudinal direction of the catheter body and the shape of the catheter body from the position at which the blood removing hole is disposed to the tip side is configured with a reduced diameter part having a small cross-sectional area and a continuous large diameter part with a large cross-sectional area. Further, (Patent Literature 5) discloses a balloon catheter configured with a catheter tube consisting of an inner tube and an outer tube and a balloon, wherein the inner tube has a first lumen with an open tip and the outer tube has the inner tube inserted therethrough and the tip thereof is disposed in a slightly more receded position than the tip of the inner tube, wherein a second lumen is formed between the inner surface of the outer tube and the outer surface of the inner tube and the tip of which communicates with the rear end part in the balloon into which a liquid for inflating the balloon flows.

### CITATION LIST

Patent Literature
Patent Literature 1: JP Patent Publication (Kokai) No. 2008-086785A
Patent Literature 2: JP Patent Publication (Kohyo) No. 2006-521177A
Patent Literature 3: JP Patent Publication (Kohyo) No. 2008-504897A
Patent Literature 4: JP Patent Publication (Kokai) No. 2001-340466A
Patent Literature 5: JP Patent Publication (Kokai) No. 7-328124A (1995)

### SUMMARY OF INVENTION

### Technical Problem

The oily contrast agent (Lipiodol) comprising an anticancer agent described above provides a transient action of the anticancer agent and cannot be expected for the controlled-releasing effect. The conventional embolic substances and preparations for forming emboli also need to be improved in the performance. More specifically, there is a demand for a preparation for forming emboli capable of retaining and controlled-releasing an anticancer agent, occluding a blood vessel quickly, unlikely to be washed out and having a controlled decomposition time, however such a preparation has not yet been developed.

Thus, the present invention, in view of the above conventional circumstances, has an object of providing a preparation for forming emboli highly safe in a living body and capable of retaining and controlled-releasing an anticancer agent, occluding a blood vessel when injected into the blood vessel, unlikely to be washed out and having a controlled decomposition time (i.e., occludes a blood vessel for a while and quickly decomposes to prevent the necrosis of the entire tissues when the function is completed).

As for the microcatheters, the conventional microcatheters having a plural number of lumens are to avoid the mixture of agents by supplying each agent to the individual lumen. Alternatively, one of the plural number of lumens is used to dilate a blood vessel by inflating a balloon and functions independently of the remaining lumens.

On the other hand, when a plural number of agents are administered, those plural number of agents separately managed before administration and never mixed with each other may need to be in a mixed condition at the time of arrival at a blood vessel and may further need to be reacted with each other. However, the conventional microcatheter failed to respond to a method for administering a plural number of agents in a mixed condition as described above.

Thus, the present invention has an object of providing a novel microcatheter wherein a plural number of agents are injected separately, mixed immediately before being released from the catheter and administered to a blood vessel in a homogeneously mixed condition. This microcatheter is, particularly, used suitably as a catheter for supplying the preparation for forming emboli according to the present invention.

### Solution to Problem

For solving the above problem, the present inventors conducted extensive studies and found that when a phenolic hydroxyl group-modified polymer having a predetermined structure is oxidatively polymerized by a peroxidase and the like, a gel capable of occluding a blood vessel is formed, whereby the invention on the preparation for forming emboli is accomplished.

As for the microcatheter, the present inventors found that the above problem is solved by forming a mixing space at the tip of a catheter for mixing agents supplied via separate lumens (an inner cavity and an outer cavity), whereby the present invention is accomplished.

More specifically, the summary of the present invention is as follows.
(1) A preparation for forming emboli, comprising

a phenolic hydroxyl group-modified polymer represented by the following formula (1):

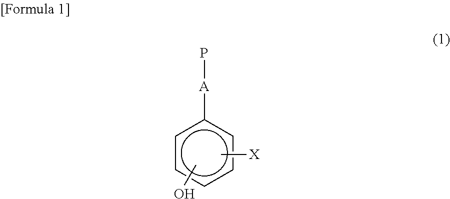

wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group,

a solution comprising at least one selected from a peroxidase, a laccase, a tyrosinase, a catalase and an iron porphyrin complex and

a solution comprising hydrogen peroxide.
(2) A preparation for forming emboli, comprising

a first solution comprising a phenolic hydroxyl group-modified polymer represented by the following formula (1):

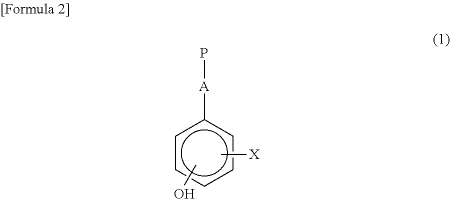

wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group and at least one selected from a peroxidase, a laccase, a tyrosinase, a catalase and an iron porphyrin complex and

a second solution comprising hydrogen peroxide.
(3) A preparation for forming emboli, comprising

a phenolic hydroxyl group-modified polymer represented by the following formula (1):

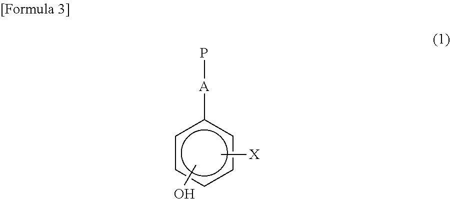

wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group, at least one selected from a peroxidase, a laccase, a tyrosinase, a catalase and an iron porphyrin complex and at least one selected from a glucose oxidase, a choline oxidase, an amino acid oxidase, an alcohol oxidase, a pyruvate oxidase and a cholesterol oxidase.
(4) The preparation for forming emboli according to any of the above (1) to (3), wherein the biocompatible polymer is a gelatin, a pectin, an albumin, a hyaluronic acid, an amylopectin, a chitosan, an alginic acid, a modified polyvinyl alcohol having a carboxyl group, a carboxymethyl cellulose or a collagen.
(5) The preparation for forming emboli according to any of the above (1) to (3), wherein the phenolic hydroxyl group-modified polymer represented by the formula (1) is a sugar beet-derived pectin having a ferulic acid group.
(6) The preparation for forming emboli according to any of the above (1) to (5) comprising, in addition to the phenolic hydroxyl group-modified polymer represented by the formula (1), a different phenolic hydroxyl group-modified polymer represented by the formula (1).
(7) The preparation for forming emboli according to any of the above (1) to (5) comprising, in addition to the phenolic hydroxyl group-modified polymer represented by the formula (1), aniline or a derivative thereof.
(8) The preparation for forming emboli according to any of the above (1) to (7), further comprising an agent.
(9) The preparation for forming emboli according to the above (8), wherein the agent is an anticancer agent.
(10) A microcatheter having a tubular catheter body that opens at a distal end,

wherein an inner cavity and an outer cavity coaxially partitioned by a partition wall are formed inside the catheter body, and

the inner cavity and the outer cavity each opens at a distal end side of the catheter body, and the partition wall is disposed back from the distal end toward a proximal end side, and thereby a mixing space for mixing liquids supplied to the inner cavity and the outer cavity respectively is formed between the opening of the inner cavity and the distal end of the catheter body.
(11) The microcatheter according to the above (10)

wherein the inner cavity is for supplying either one of a first solution or a second solution, the first solution comprising a phenolic hydroxyl group-modified polymer represented by the following formula (1):

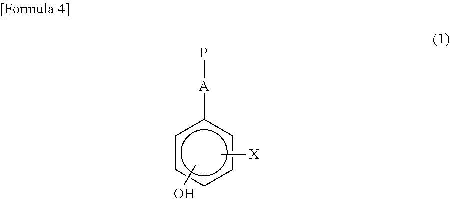

wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group and at least one selected from a peroxidase, a laccase, a tyrosinase, a catalase and an iron porphyrin complex and

the second solution comprising hydrogen peroxide, and

the outer cavity is for supplying the other.
(12) The microcatheter according to the above (11), wherein the biocompatible polymer is a gelatin, a pectin, an albumin, a hyaluronic acid, an amylopectin, a chitosan, an alginic acid, a modified polyvinyl alcohol having a carboxyl group, a carboxymethyl cellulose or a collagen.
(13) The microcatheter according to the above (11), wherein the phenolic hydroxyl group-modified polymer represented by the formula (1) is a sugar beet-derived pectin having a ferulic acid group.

The present Description encompasses the disclosed contents described in Japanese Patent Application No. 2014-213293, which is the basis of priority of the present application.

### Advantageous Effects of Invention

The preparation for forming emboli according to the present invention is a preparation (injectable gel) which is a liquid before the injection into a living body but quickly gelled after the injection into the living body. The preparation can form a gel in a blood vessel to the same shape as the blood vessel without leaving any gaps, hence is less likely to be washed out. The decomposition time of the gel is also controlled depending on the type of a biocompatible polymer and the concentration of each component, whereby the safety in a living body is assured. Further, when the preparation comprises an agent such as an anticancer agent in advance, the agent retention by the gel and the controlled-release of the agent from the gel are enabled.

According to the microcatheter of the present invention, a plural number of agents are separately injected to each of the inner cavity and outer cavity, mixed immediately before being released from the catheter and administered into a blood vessel and the like in a homogeneously mixed condition.

In particular, the microcatheter of the present invention is preferably used as a suitable catheter for administering the preparation for forming emboli (injectable gel) of the present invention as described above to occlude a blood vessel by mixing the first solution comprising a phenolic hydroxyl group-modified polymer such as a sugar beet-derived pectin and a peroxidase and the second solution comprising hydrogen peroxide and reacting the mixture upon the injection into a blood vessel to be gelled quickly.

### BRIEF DESCRIPTION OF DRAWINGS

FIG. 1 is a drawing showing a histopathological image (alcian blue stained) on Day 0 from intraportal administration of the preparation for forming emboli according to the present invention.

FIG. 2 is a drawing showing a histopathological image (alcian blue stained) on Day 1 from intraportal administration of the preparation for forming emboli according to the present invention.

FIG. 3 is a drawing showing a histopathological image (alcian blue stained) on Day 3 from intraportal administration of the preparation for forming emboli according to the present invention.

FIG. 4 is a drawing showing a histopathological image (alcian blue stained) on Day 7 from intraportal administration of the preparation for forming emboli according to the present invention.

FIG. 5 is a graph showing the AST and ALT changes after intraportal administration of the preparation for forming emboli according to the present invention.

FIG. 6 is a graph showing the γGTP and total bilirubin changes after intraportal administration of the preparation for forming emboli according to the present invention.

FIG. 7 is a drawing illustrating an embodiment of the microcatheter of the present invention.

FIG. 8 is an enlarged cross-sectional view of the microcatheter tip.

### DESCRIPTION OF EMBODIMENTS

Hereinafter, the present invention is described in detail.

The preparation for forming emboli according to the present invention comprises a solution comprising a phenolic hydroxyl group-modified polymer represented by the following formula (1):

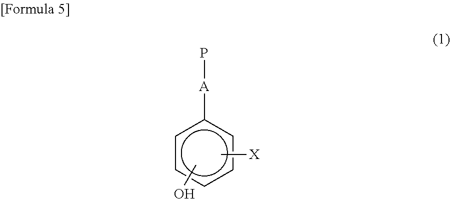

wherein P is a biocompatible polymer, A is a single bond or an —OCO—C₂-C₄-alkenylene group, a —CONH—C₁-C₄-alkylene group or an —HNCO—C₁-C₄-alkylene group (written from the side bonding to P), and X is hydrogen or a C₁-C₃-alkoxy group,
a solution comprising at least one selected from a peroxidase, a laccase, a tyrosinase, a catalase and an iron porphyrin complex and a solution comprising hydrogen peroxide. When these solutions are mixed, hydrogen peroxide serves as an oxidizing agent at the phenol moiety of the phenolic hydroxyl group-modified polymer and causes an oxidative polymerization reaction by a peroxidase, a laccase, a tyrosinase, a catalase or an iron porphyrin complex, whereby the phenolic hydroxyl group-modified polymers are crosslinked each other and quickly gelled. Such a reaction occurs in a living body and is substantially non-toxic, hence advantageous. The preparation for forming emboli according to the present invention can be used as an “injectable gel”, which is a liquid before the injection into a living body but quickly gelled after the injection into the living body.

Example of the —OCO—C₂-C₄-alkenylene group in the above formula (1) include straight chained or branched groups such as —OCO—CH═CH— groups. Further, examples of the —CONH—C₁-C₄-alkylene group include straight chained or branched groups such as —CONH—CH₂— groups and —CONH—CH₂CH₂— groups, and examples of the —HNCO—C₁-C₄-alkylene group include straight chained or branched groups such as —HNCO—CH₂— groups and —HNCO—CH₂CH₂— groups. Further, the OH group and X may bond to any position of the benzene ring and examples of the C₁-C₃-alkoxy group corresponding to X include methoxy groups, ethoxy groups, propoxy groups and isopropoxy groups.

The gelation starts as soon as hydrogen peroxide contacts the phenolic hydroxyl group-modified polymer and at least one selected from a peroxidase, a laccase, a tyrosinase, a catalase and an iron porphyrin complex (hereinafter sometimes referred to as “peroxidase, etc.”), and thus in the preparation for forming emboli according to the present invention, the phenolic hydroxyl group-modified polymer and the peroxidase, etc. need to be separated from hydrogen peroxide and these components need to be mixed immediately before the injection into a blood vessel. On the other hand, the phenolic hydroxyl group-modified polymer and the peroxidase, etc. can be mixed in advance without any problem and thus it is preferable, from a perspective of the preparation operability, that the preparation for forming emboli according to the present invention be provided as a kit consisting of the first solution comprising the phenolic hydroxyl group-modified polymer and the peroxidase, etc. and the second solution comprising hydrogen peroxide. Needless to say, the preparation is not limited thereto and may be constituted with three or more solutions.

Any biocompatible polymer is applicable as long as, when used to a living body, it is not toxic in an amount used, chemically inactive and nonimmune. Preferable examples include gelatins, pectins, albumins, hyaluronic acids, amylopectins, chitosans, alginic acids, modified polyvinyl alcohols having a carboxyl group, carboxymethyl celluloses or collagens. The introduction of a side chain comprising the phenol moiety represented by the formula (1) to these biocompatible polymers can be carried out by a typically known method. More specifically, 3-(p-hydroxyphenyl)propionic acid and tyramine comprising the phenol moiety are reacted and then introduced respectively to the amino group and carboxyl group of the biocompatible polymer by the typical reaction called carbodiimide chemistry. Examples of the biocompatible polymer having an amino group include chitosans, and examples of the biocompatible polymer having a carboxyl group include gelatins, pectins, albumins, hyaluronic acids, amylopectins, alginic acids, modified polyvinyl alcohols having a carboxyl group, carboxymethyl celluloses and collagens.

Particularly, a sugar beet-derived pectin is preferably used as the phenolic hydroxyl group-modified polymer represented by the formula (1). The sugar beet-derived pectin is a polysaccharide extracted from sugar beet and purified. Specifically, the pectin can be produced by suspending in water sugar beet remain (sugar beet pulp), i.e. the residue when sucrose is produced from sugar beet, and sugar beet as starting materials and subjecting the suspension to acidic hot water extraction under the acidic condition of pH 1 to 7 at the temperature of 50° C. to 120° C. but the production method is not limited thereto.

The sugar beet-derived pectin has neutral sugar and, as shown in the following formula, ferulic acid is ester-bonded to the neutral sugar. The sugar beet-derived pectin undergoes an oxidative polymerization reaction by the peroxidase, etc. using hydrogen peroxide as an oxidizing agent and the phenol moieties are crosslinked thereby quickly forming a gel.

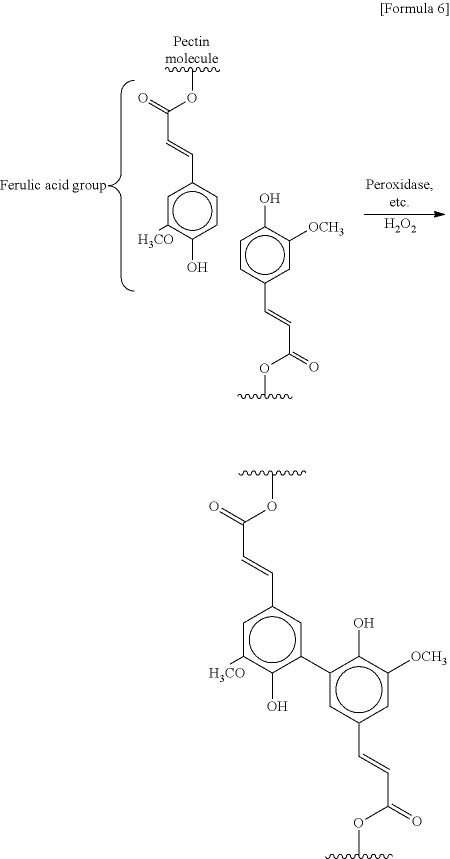

The peroxidase, laccase, tyrosinase, catalase and iron porphyrin complex described above are not particularly limited and those naturally occurred and synthesized are applicable. For example, the peroxidase can be suitably selected for use from those derived from animals such as human and cow, those derived from plants such as horseradish and those derived from microorganisms such as bacteria and mold. Also applicable are those prepared by gene recombination technology such as introducing a peroxidase gene of a microorganism, etc. to a microorganism, etc. such as E. coli.

The concentrations of the phenolic hydroxyl group-modified polymer, the peroxidase, etc. and hydrogen peroxide vary depending on the specific type and molecular weight of each component and intended gelation time and are not particularly limited. As an example, when the preparation for forming emboli is constituted with the first solution comprising the sugar beet-derived pectin and a peroxidase and the second solution comprising hydrogen peroxide, it is preferable that the concentration of the sugar beet-derived pectin be 0.5 to 10 wt % and the concentration of peroxidase be 0.5 to 200 unit/ml. It is also preferable that, in the second solution, the concentration of hydrogen peroxide be 0.1 to 200 mmol/l. It is further preferable that the first and the second solutions be mixed and gelled in a ratio of 1:1-9:1 (volume ratio). Note that it is preferable that, from a perspective of injecting into a blood vessel, the viscosity of the first solution be approximately 1 to 200 mPa·s at 37° C. Note that the relationship of the peroxidase activity and the weight thereof is 210 unit/mg. The enzyme amount needed to oxidize 1 μmol of guaiacol per minute at pH 7.0, 25° C. is defined as 1 unit.

In another embodiment, the preparation for forming emboli according to the present invention can use, in place of hydrogen peroxide, at least one selected from a glucose oxidase, a choline oxidase, an amino acid oxidase, an alcohol oxidase, a pyruvate oxidase and a cholesterol oxidase (hereinafter sometimes referred to as “glucose oxidase, etc.”) and be provided as a single liquid preparation comprising the phenolic hydroxyl group-modified polymer represented by the formula (1), the peroxidase, etc. and the glucose oxidase, etc. The phenolic hydroxyl group-modified polymer used herein can be the same polymer as above. When the preparation for forming emboli consisting of a single liquid is injected into a living body, hydrogen peroxide is produced by the glucose oxidase etc. in the living body. For example, hydrogen peroxide produced when a glucose oxidase reacts to a glucose in a living body serves as an oxidizing agent whereby the phenolic hydroxyl group-modified polymers are crosslinked and gelled. This preparation does not need to mix two liquids and the gelation is achieved only by injecting the single liquid into a living body, hence advantageous.

For the glucose oxidase, etc., those naturally occurred and synthesized can be used without being particularly limited. For example, commercial glucose oxidases, which are enzymes produced by microorganisms such as Aspergillus niger and Penicillium chrysogenum, can be used as the glucose oxidase.

In this embodiment, each concentration of the phenolic hydroxyl group-modified polymer, the peroxidase, etc. and the glucose oxidase, etc. in the preparation varies depending on the specific type and molecular weight of each component and intended gelation time and is not particularly limited. As an example, when the sugar beet-derived pectin is adopted as the phenolic hydroxyl group-modified polymer and a peroxidase and a glucose oxidase are further used, it is preferable that, in the preparation, the concentration ranges of the sugar beet-derived pectin be 0.5 to 5.0 wt %, the concentration of the peroxidase be 0.5 to 200 unit/ml and the concentration of the glucose oxidase be 0.5 to 200 unit/ml. Note that the relationship of the glucose oxidase activity and the weight thereof is 241 unit/mg. The enzyme amount needed to oxidize 1 μmol of β-D-glucose to D-gluconolactone and hydrogen peroxide per minute at pH 5.1, 35° C. is defined as 1 unit.

Further, the preparation for forming emboli according to the present invention may comprise as necessary, in addition to the phenolic hydroxyl group-modified polymer represented by the formula (1), a different phenolic hydroxyl group-modified polymer represented by the formula (1). Such a different phenolic hydroxyl group-modified polymer can be used as a single solution of the different phenolic hydroxyl group-modified polymer or in a solution together with the other phenolic hydroxyl group-modified polymer or the peroxidase, etc. and the glucose oxidase, etc. as long as the solution is separated from hydrogen peroxide. These different phenolic hydroxyl group-modified polymers are mainly used to adjust the gel decomposition time. For example, the sugar beet-derived pectin has a comparatively slow decomposition rate due to the lack of a decomposition enzyme for the sugar beet-derived pectin in a living body. Then, for example, a more easily decomposable gelatin is mixed in a range of the sugar beet derived-pectin:gelatin=1:9 to 9:1 (weight ratio) whereby not only the sugar beet-derived pectin but also the sugar beet-derived pectin and the gelatin are crosslinked each other to form a gel. In this gel, the crosslinked structure is broken due to the faster gelatin decomposition and the gel decomposition time can thus be cut down by about 50 to 200% when compared with the gel composed of the sugar beet-derived pectin alone.

Further, the preparation for forming emboli according to the present invention may comprise as necessary, in addition to the phenolic hydroxyl group-modified polymer represented by the formula (1), aniline or a derivative thereof. The aniline derivatives applicable are compounds in which the aniline backbone has an alkyl group, an amino group, a cyano group, a sulfonic acid group, a hydroxy group or a carboxyl group and specific examples include aminophenol and diaminobenzene but are not limited thereto. Aniline or a derivative thereof reacts to the phenolic hydroxyl group-modified polymer by an oxidative polymerization reaction in the presence of the above peroxidase, etc. and hydrogen peroxide or the glucose oxidase, etc. thereby forming a gel. When the phenolic hydroxyl group-modified polymer and aniline or a derivative thereof are used in combination, the mixing ratio can suitably be set so as to achieve desired gelation time and may be, for example, the phenolic hydroxyl group-modified polymer:aniline or a derivative thereof=1:9 to 9:1 (weight ratio).

The preparation for forming emboli according to the present invention can further comprise various agents such as an anticancer agent. These agents are retained in the gel occluding a blood vessel and can be controlled-released as the gel decomposes. Accordingly, the preparation can be used as a drug delivery system (DDS). Examples of the anticancer agent include conventionally known substances such as epirubicin and cisplatin. Note that the “anticancer agent” used herein may be any agent that intends to suppress the growth of malignant tumors (cancers) and means to encompass not only anticancer agents in a narrow sense but also the so-called cancer control agents. The concentration of the agent in the gel can be suitably set depending on the type of agents and the target disease.

The preparation for forming emboli according to the present invention can further comprise various additives as necessary. Specific examples of the additive include pH adjusters such as 4-(2-hydroxyethyl)-1-piperazine ethanesulfonic acid.

The preparation for forming emboli according to the present invention can be applicable to various mammals. Examples of the mammal include human, mouse, rat, rabbit, dog, cat, cow, horse, pig and monkey who are expected to have a disease treated when a blood vessel is occluded. The injection site of the preparation for forming emboli is not particularly limited and can be determined depending on the target disease, and preferable examples include the hepatic artery into which the preparation can be injected using a microcatheter. For example, the microcatheter according to the present invention as described below is preferably used as the catheter for injecting the above preparation for forming emboli.

The microcatheter according to the present invention is described next in detail.

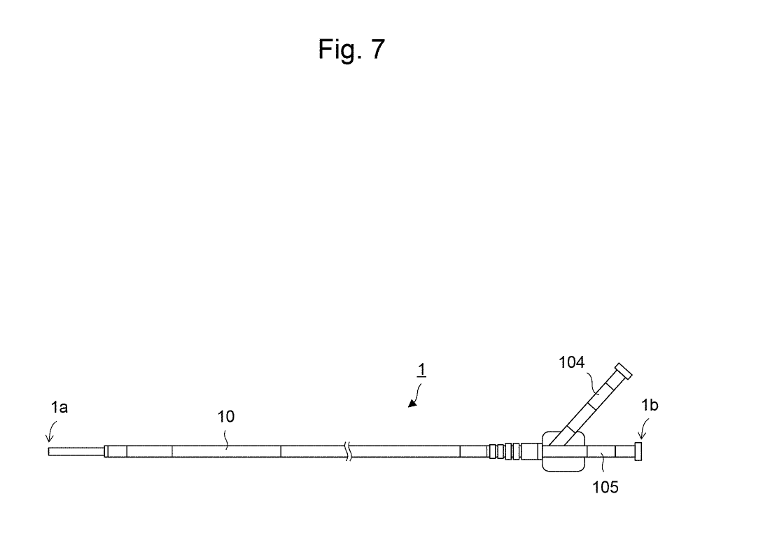

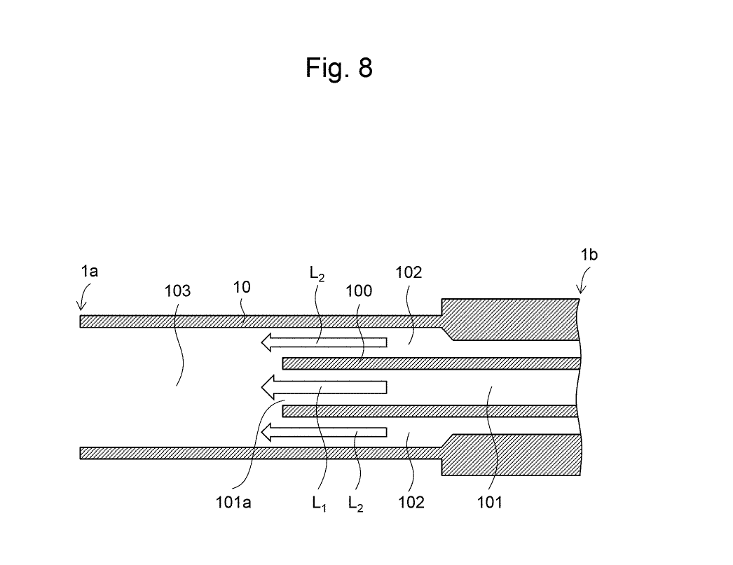

An embodiment of the microcatheter of the present invention is described in reference to FIG. 7 and FIG. 8. The microcatheter 1 of the present invention has, as illustrated in FIG. 7, a tubular catheter body 10 that opens at a distal end 1a.

The catheter body 10 can be made of various materials such as a stainless steel or a plastic and is typically flexible. It is also preferable to be compatible with a target site such as a blood vessel and may be coated as necessary to impart hydrophilicity and antibacterial properties to the surface. Also, for the purpose of improving the visibility of the microcatheter under fluoroscopy, a radiopaque tip may be installed on the outer side or inner side of the tip (the tip part including the distal end 1a) of the catheter body 10.

Inside the catheter body 10, as illustrated in FIG. 8, an inner cavity 101 and an outer cavity 102 coaxially partitioned by a partition wall 100 are formed. The inner cavity 101 and the outer cavity 102 each opens at the distal end 1a side of the catheter body 10, and the partition wall 100 is disposed back from the distal end 1a of the catheter body 10 toward a proximal end 1b side, and thereby a mixing space 103 for mixing liquids L₁, L₂ supplied to the inner cavity 101 and the outer cavity 102 respectively is formed between an opening 101a of the inner cavity 101 and the distal end 1a of the catheter body 10.

The size of the mixing space 103 may be a size enough for thoroughly mixing the supplied L₁, L₂ before released from the distal end 1a and is not particularly limited. As an example, when the first solution comprising the phenolic hydroxyl group-modified polymer and the peroxidase, etc. and the second solution comprising hydrogen peroxide as described later are supplied to the inner cavity 101 and the outer cavity 102 respectively at a pressure of about 0.1 ml/min to 1.0 ml/min and, the mixing space 103 may have a diameter of about 1.5 to 5.0 mm and a length of about 5 to 10 mm for achieving a homogeneous mixing condition therein. The above pressure can be about 1.5 ml/sec at the time of angiography.

The liquids L₁, L₂ are supplied to the inner cavity 101 and the outer cavity 102 respectively by connecting syringes to connecting parts 104, 105 located at the proximal end 1b side. Alternatively, a micropump may be connected to control the amounts of liquids to be injected into the catheter and/or the injection pressure. Further, a balloon may be provided on the outer side of the catheter body 10 as necessary to dilate a target tube such as a blood vessel.

The tube to which the microcatheter 1 is introduced is any tube located inside or outside the body of mammals such as human. Examples include veins, arteries, esophagus, urethra and intestines. The organs and tissues to be treated using the microcatheter 1 include any organs and tissues of mammals and specific examples include liver, heart, lungs, brain, kidneys, bladder, intestines, stomach, pancreas, ovaries and prostate glands.

The liquids L₁, L₂ administered using the microcatheter 1 are not particularly limited but the microcatheter of the present invention is preferably used for liquids that need to be supplied separately via the inner cavity 101 and the outer cavity 102 and mixed, or may be reacted, immediately before released from the microcatheter 1. Examples of the liquid include pharmaceutically active compounds such as anticancer agents, antiinflammatory agents, carriers, solvents, polymers, proteins, cells, contrast agents, DNA, genes/vectors, antiangiogenic agents, antioxidants, oxidizing agents, antibacterial agents and anesthetic agents.

Particularly, the microcatheter of the present invention is preferably used for administering a preparation (injectable gel) which is a liquid before the injection into a living body but quickly gelled after the injection into the living body. This preparation can form a gel in a blood vessel to the same shape as the blood vessel and is used as the preparation for forming emboli for occluding a blood vessel. The gel formed is decomposed after a given time has passed.

An embodiment of the preparation for forming emboli is the preparation for forming emboli according to the present invention as described above. More specifically, the preparation comprises a first solution comprising the phenolic hydroxyl group-modified polymer represented by the following formula (1):

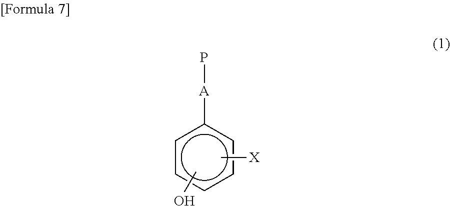

wherein P is a biocompatible polymer, A is a single bond or an —OCO—C₂-C₄-alkenylene group, a —CONH—C₁-C₄-alkylene group or an —HNCO—C₁-C₄-alkylene group (written from the side bonding to P), and X is hydrogen or a C₁-C₃-alkoxy group
and at least one selected from a peroxidase, a laccase, a tyrosinase, a catalase and an iron porphyrin complex (hereinafter sometimes referred to as “peroxidase, etc.”) and a second solution comprising hydrogen peroxide. Either one of the first solution or the second solution is supplied to the inner cavity 101 of the catheter body 10, the other of the first solution or the second solution is supplied to the outer cavity 102 and the solutions are homogeneously mixed in the mixing space 103 and then released. When mixed, hydrogen peroxide serves as an oxidizing agent at the phenol moiety of the phenolic hydroxyl group-modified polymer and causes an oxidative polymerization reaction by the peroxidase, etc., whereby the phenolic hydroxyl group-modified polymers are crosslinked each other and quickly gelled. Such a reaction occurs in a living body and is substantially non-toxic, hence advantageous.

Example of the —OCO—C₂-C₄-alkenylene group in the above formula (1) include straight chained or branched groups such as —OCO—CH=CH— groups. Further, examples of the —CONH—C₁-C₄-alkylene group include straight chained or branched groups such as —CONH—CH₂— groups and —CONH—CH₂CH₂— groups, and examples of the —HNCO—C₁-C₄-alkylene group include straight chained or branched groups such as —HNCO—CH₂— groups and —HNCO—CH₂CH₂— groups. Further, the OH group and X may bond to any position of the benzene ring and examples of the C₁-C₃-alkoxy group corresponding to X include methoxy groups, ethoxy groups, propoxy groups and isopropoxy groups.

The gelation starts as soon as hydrogen peroxide contacts the phenolic hydroxyl group-modified polymer and the peroxidase, etc. and thus in the preparation for forming emboli, the phenolic hydroxyl group-modified polymer and the peroxidase, etc. need to be separated from hydrogen peroxide and these components need to be mixed immediately before the injection into a blood vessel. On the other hand, the phenolic hydroxyl group-modified polymer and the peroxidase, etc. can be mixed in advance without any problem and thus, from a perspective of the handling of the preparation, the preparation for forming emboli is provided as a kit consisting of the first solution comprising the phenolic hydroxyl group-modified polymer and the peroxidase, etc. and the second solution comprising hydrogen peroxide.

Any biocompatible polymer is applicable as long as, when used to a living body, it is not toxic in an amount used, chemically inactive and nonimmune. Preferable examples include gelatins, pectins, albumins, hyaluronic acids, amylopectins, chitosans, alginic acids, modified polyvinyl alcohols having a carboxyl group, carboxymethyl celluloses or collagens. The introduction of a side chain comprising the phenol moiety represented by the formula (1) to these biocompatible polymers can be carried out by a typically known method. More specifically, 3-(p-hydroxyphenyl)propionic acid and tyramine comprising the phenol moiety are reacted and then introduced respectively to the amino group and carboxyl group of the biocompatible polymer by the typical reaction called carbodiimide chemistry. Examples of the biocompatible polymer having an amino group include chitosans, and examples of the biocompatible polymer having a carboxyl group include gelatins, pectins, albumins, hyaluronic acids, amylopectins, alginic acids, modified polyvinyl alcohols having a carboxyl group, carboxymethyl celluloses and collagens.

Particularly, a sugar beet-derived pectin is preferably used as the phenolic hydroxyl group modified polymer. The sugar beet-derived pectin is a polysaccharide extracted from sugar beet and purified. Specifically, the pectin can be produced by suspending in water sugar beet remain (sugar beet pulp), i.e. the residue when sucrose is produced from sugar beet, and sugar beet as starting materials and subjecting the suspension to acidic hot water extraction under the acidic condition of pH 1 to 7 at 50° C. to 120° C. but the production method is not limited thereto.

The sugar beet-derived pectin has neutral sugar and, as shown in the following formula, ferulic acid is ester-bonded to the neutral sugar. The sugar beet-derived pectin undergoes an oxidative polymerization reaction by the peroxidase, etc. using hydrogen peroxide as an oxidizing agent and the phenol moiety crosslinks thereby quickly forming a gel.

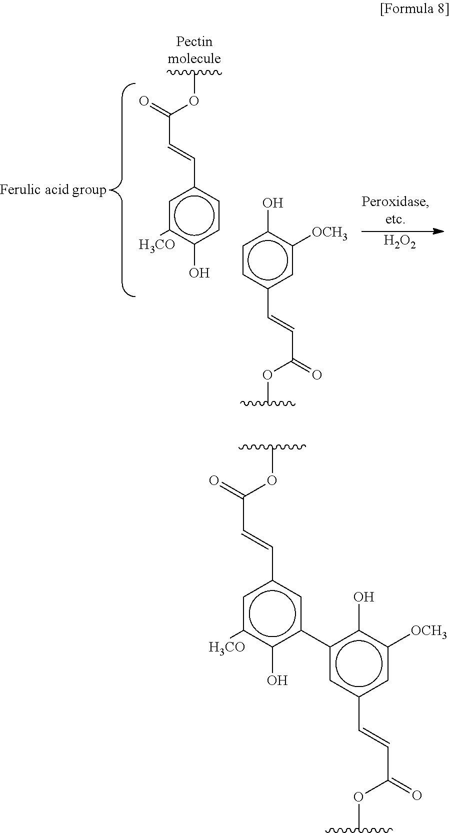

The peroxidase, laccase, tyrosinase, catalase and iron porphyrin complex described above are not particularly limited and those naturally occurred and synthesized are applicable. For example, the peroxidase can be suitably selected for use from those derived from animals such as human and cow, those derived from plants such as horseradish and those derived from microorganisms such as bacteria and mold. Also applicable are those prepared by gene recombination technology such as introducing a peroxidase gene of a microorganism to a microorganism such as E. coli.

Each concentration of the phenolic hydroxyl group modified polymer, the peroxidase, etc. and hydrogen peroxide varies depending on the specific type and molecular weight of each component and intended gelation time and is not particularly limited. As an example, when the preparation for forming emboli is constituted with the first solution comprising the sugar beet-derived pectin and a peroxidase and the second solution comprising hydrogen peroxide, it is preferable that the concentration of the sugar beet-derived pectin be 0.5 to 10 wt % and the concentration of peroxidase be 0.5 to 200 unit/ml. It is also preferable that, in the second solution, the concentration of hydrogen peroxide be 0.1 to 200 mmol/l. It is further preferable that the first and the second solutions be mixed and gelled in a ratio of 1:1-9:1 (volume ratio). Note that it is preferable that, from a perspective of injecting into a blood vessel, the viscosity of the first solution be approximately 1 to 200 mPa·s at 37° C. Note that the relationship of the peroxidase activity and the weight thereof is 210 unit/mg. The enzyme amount needed to oxidize 1 μmol of guaiacol per minute at pH 7.0, 25° C. is defined as 1 unit.

Further, the preparation for forming emboli may comprise as necessary, in addition to the phenolic hydroxyl group-modified polymer represented by the formula (1), a different phenolic hydroxyl group-modified polymer represented by the formula (1). Such a different phenolic hydroxyl group-modified polymer can be used in a solution together with the other phenolic hydroxyl group-modified polymer or the peroxidase, etc. as long as the solution is separated from hydrogen peroxide. These different phenolic hydroxyl group-modified polymers are mainly used to adjust the gel decomposition time. For example, the sugar beet-derived pectin has a comparatively slow decomposition rate due to the lack of a decomposition enzyme for the sugar beet-derived pectin in a living body. Then, for example, a more easily decomposable gelatin is mixed in a range of the sugar beet derived-pectin:gelatin=1:9 to 9:1 (weight ratio) whereby not only the sugar beet-derived pectin but also the sugar beet-derived pectin and the gelatin are crosslinked each other to form a gel. In this gel, the crosslinked structure is broken due to the faster gelatin decomposition and the gel decomposition time can thus be cut down by about 50 to 200% when compared with the gel composed of the sugar beet-derived pectin alone.

Further, the preparation for forming emboli may comprise as necessary, in addition to the phenolic hydroxyl group-modified polymer represented by the formula (1), aniline or a derivative thereof. The aniline derivatives applicable are compounds in which the aniline backbone has an alkyl group, an amino group, a cyano group, a sulfonic acid group, a hydroxy group or a carboxyl group and specific examples include aminophenol and diaminobenzene but are not limited thereto. Aniline or a derivative thereof reacts to the phenolic hydroxyl group-modified polymer by an oxidative polymerization reaction in the presence of the above peroxidase, etc. and hydrogen peroxide thereby forming a gel. When the phenolic hydroxyl group-modified polymer and aniline or a derivative thereof are used in combination, the mixing ratio can suitably be set so as to achieve desired gelation time and may be, for example, the phenolic hydroxyl group-modified polymer:aniline or a derivative thereof=1:9 to 9:1 (weight ratio).

The preparation for forming emboli can further comprise various agents such as pharmaceutically active compounds such as anticancer agents, antiinflammatory agents, etc., carriers, solvents, polymers, proteins, cells, contrast agents, DNA, genes/vectors, antiangiogenic agents, antioxidants, oxidizing agents, antibacterial agents and anesthetic agents. These agents are retained in the gel occluding a blood vessel and can be controlled-released as the gel decomposes. Accordingly, the preparation can be used as a drug delivery system (DDS). Examples of the anticancer agent include conventionally known substances such as epirubicin and cisplatin. Note that the “anticancer agent” used herein may be any agent that intends to suppress the growth of malignant tumors (cancers) and means to encompass not only anticancer agents in a narrow sense but also the so-called cancer control agents. The concentration of the agent in the gel can be suitably set depending on the type of agent and the target disease.

The preparation for forming emboli can further comprise various additives as necessary. Specific examples of the additive include pH adjusters such as 4-(2-hydroxyethyl)-1-piperazine ethanesulfonic acid.

### EXAMPLE

The present invention is described next in further detail in reference to Examples and Comparative Examples but is not limited thereto.

### Example 1

Two liquids of 0.9 ml of an aqueous solution in which the sugar beet-derived pectin and a peroxidase were dissolved and 0.1 ml of an aqueous solution of hydrogen peroxide were mixed and then a concentration at which each substance was quickly gelled was examined The results revealed that the gelation was quickly (within 10 seconds) occurred when the concentrations were 3.0 (w/v) % (sugar beet-derived pectin concentration), 10 unit/ml (peroxidase concentration) and 1 mmol/l (hydrogen peroxide concentration), respectively.

### Example 2

Next, under the assumption of intravascular administration, the gelation was confirmed using a blood vessel model with adjustable blood pressure, pulse and humidity. The blood vessel model is a closed circuit, in the middle of which tubes with a diameter smaller than 1.5 mm were manufactured using an acrylic plate assumedly representing capillary vessels, thereby making the model useful to confirm the gelation in the flowing liquid. Distilled water was circulated in this blood vessel model at a pressure of 125 mmHg.

Next, the microcatheter as illustrated in FIG. 7 and FIG. 8 was manufactured. In the microcatheter, an opening diameter at the distal end 1a was 3 mm, an opening diameter of the inner cavity 101 was 0.43 mm and a length of the mixing space 103 (the distance from the distal end 1a to the partition walls 100) was 5 mm. Note that the above microcatheter was manufactured by modifying an existing end balloon catheter (with flow channels of the inner side being a double layer structure). The manufactured microcatheter is configured in such a way that the inner cavity and the outer cavity are each independent, the first solution and the second solution passing through the inner cavity and the outer cavity respectively meet at the catheter tip and are released to outside from the catheter tip in a mixed condition.

An aqueous solution (a first solution) comprising the sugar beet-derived pectin (concentration: 3.3 wt %) and a peroxidase (concentration: 11.1 unit/ml) was flowed into the inner cavity of the microcatheter at a rate of 0.9 ml/min, an aqueous solution (a second solution) comprising hydrogen peroxide (concentration 10 mmol/l) was flowed into the other outer cavity at a rate of 0.1 ml/min and 1.0 ml of the mixed solution of the first solution and the second solution was injected into the blood vessel model from the catheter tip. After injection, the mixed solution was flowed into the capillary vessels in the blood vessel model, occluded the capillary vessels and was gelled several seconds later.

### Example 3

First, an aqueous solution (a first solution) comprising the sugar beet-derived pectin (concentration: 3.3 wt %), a peroxidase (concentration: 11.1 unit/ml) and epirubicin (concentration: 5 mg/ml) as an anticancer agent and an aqueous solution (a second solution) comprising hydrogen peroxide (concentration 10 mmol/l) were prepared. Next, the first solution was flowed into the inner cavity of the same microcatheter as in Example 2 at a rate of 0.9 ml/min, the second solution was flowed into the other outer cavity at a rate of 0.1 ml/min and 1.0 ml of the mixed solution of the first solution and the second solution was released to outside from the catheter tip. After the release, the mixed solution was gelled within several seconds. The gelation time did not tend to extend when compared with the case where epirubicin was not added and it was confirmed that the gelation was almost instantaneously achieved and the agent was retained in the gel. Further, the same experiment was carried out using an anticancer cisplatin in place of epirubicin whereby the gelation time did not tend to extend as in the case of epirubicin.

### Example 4

First, 1.0 ml of an aqueous solution comprising the sugar beet-derived pectin (concentration: 3.6 wt %) and L929 fibroblast (concentration: 1.5×10⁵ cells/ml), 0.1 ml of an aqueous solution comprising a peroxidase (concentration: 12 unit/ml) and 0.1 ml of an aqueous solution comprising hydrogen peroxide (concentration 1.2 mmol/l) were prepared. Next, the aqueous solution of the peroxidase and the aqueous solution of hydrogen oxide were added to and mixed with the above aqueous solution of the sugar beet-derived pectin and L929 fibroblast. The mixed solution was gelled 10 seconds later and a gel thin film having a thickness of less than 100 μm was formed. The obtained gel thin film had the viable cells and the dead cells stained simultaneously using Cellstain (registered trademark, a cell double stain kit) and observed using a fluorescence microscope. As a result of the observation, it was confirmed that the overall cells remain substantially unaffected during the gelation process and the preparation for forming emboli according to the present invention and the gel formed with the preparation are highly safe in a living body.

### Example 5

Next, using the above microcatheter, adverse effects were confirmed on animal models when the preparation for forming emboli according to the present invention was intraarterially administered.

First, a rabbit was systemically anesthesized, subsequently laparotomized to expose the appendix and the portal vein was punctured in which the same microcatheter as in Example 2 was catheterized under fluoroscopy. An aqueous solution (a first solution) in which the sugar beet-derived pectin (concentration: 3.3 wt %) and a peroxidase (concentration: 11.1 unit/ml) were dissolved and an aqueous solution (a second solution) comprising hydrogen peroxide (concentration 10 mmol/l) were respectively connected to the microcatheter and, using a syringe pump (pressure pump), the aqueous solution of hydrogen peroxide at a rate of 0.1 ml/min and the aqueous solution in which the sugar beet-derived pectin and the peroxidase were dissolved at a rate of 0.9 ml/min were intravascularly administered as the mixed solution from the microcatheter tip. The dose was set so that the sugar beet-derived pectin was 5 ml. The intraportally administered rabbit had the blood drawn immediately after the administration (30 minutes), on Day 1, Day 3 and Day 7 and the liver was removed to carry out the blood biochemical examination and the histopathological examination.

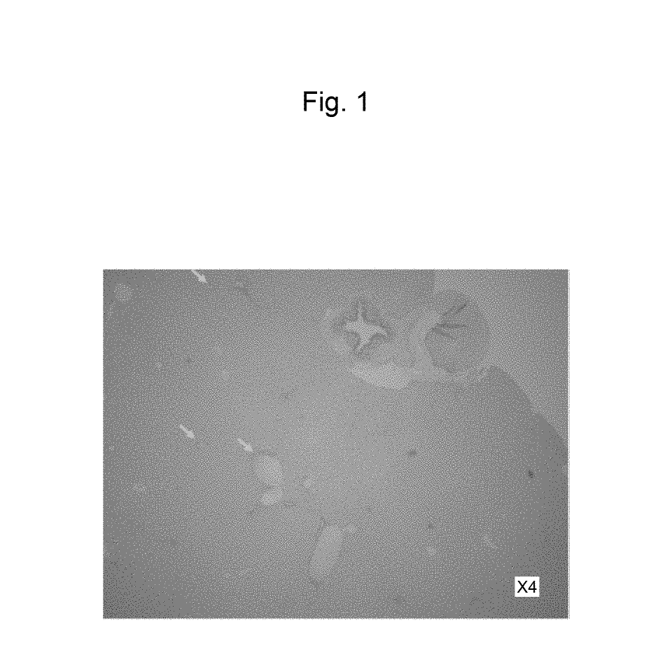

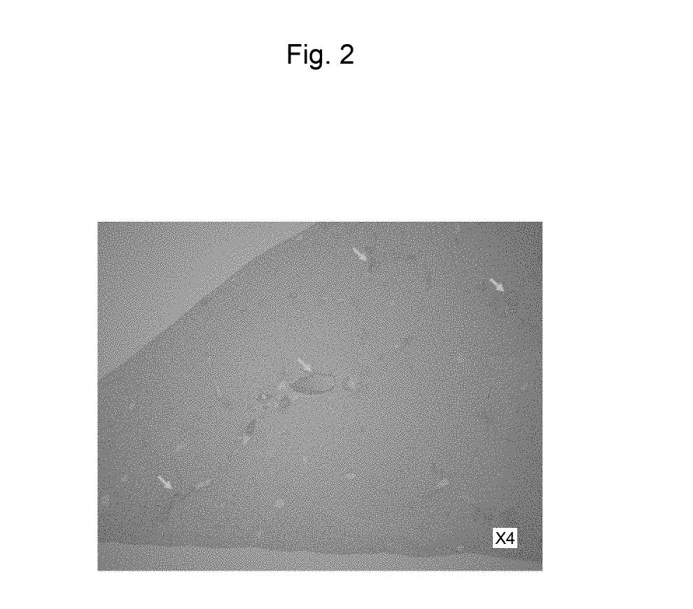

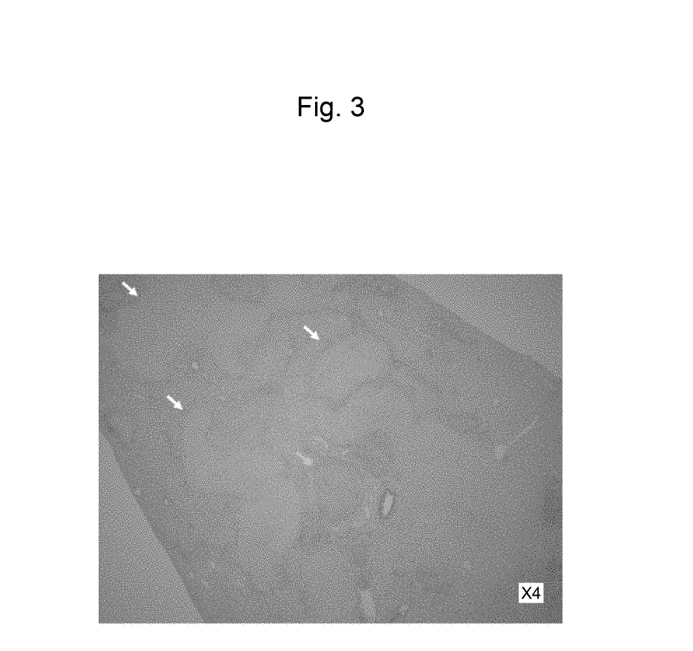

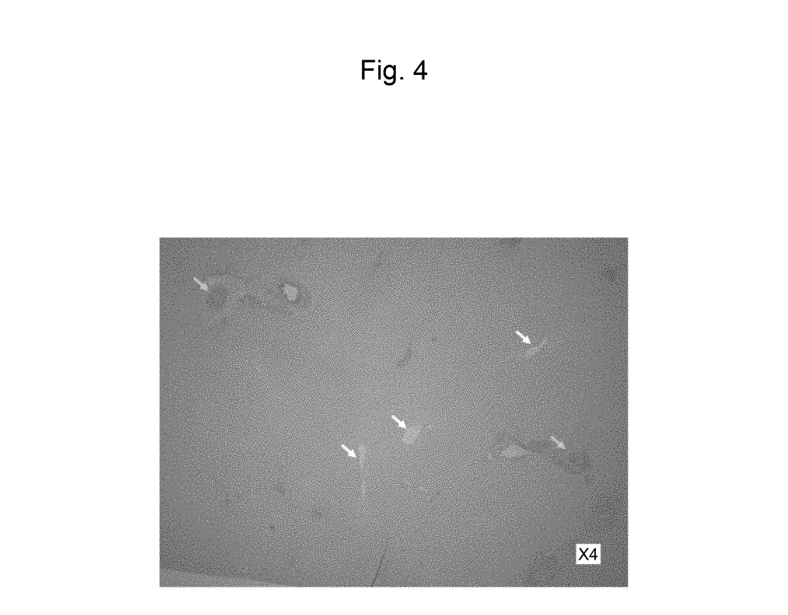

After intraportal administration of the preparation for forming emboli, the visible findings of the liver on Day 3 showed the color changed to reddish brown on the right lobe and spotty white color changes presumably caused by the formed gel or the inflammation associated therewith were found. FIGS. 1 to 4 show the time-dependent histopathological images (alcian blue stained). On Day 0 (FIG. 1), Day 1 (FIG. 2), Day 3 (FIG. 3) and Day 7 (FIG. 4) from the injection of the preparation for forming emboli, the gel injected into the portal vein and remained in the portal vein as the embolic substance was stained blue, respectively (FIGS. 1 to 4, gray arrows). The preparation for forming emboli was found to have flowed in to the peripheral portal veins immediately after the injection and the gel was identifiable in the portal veins on the peripheral side on Day 1. On Day 3, an image was observed in which the gel was washed away from the center side to the peripheral side where liver damage associated with an embolus was notable particularly on Day 3 (FIG. 3, white arrows). However, on Day 7, the gel was remained in the portal vein but the inflammation was alleviated. In the histopathology from Day 0 to Day 7, the gel drain into the hepatic artery was not detected or death of the rabbit related to the intraportal administration did not occur.

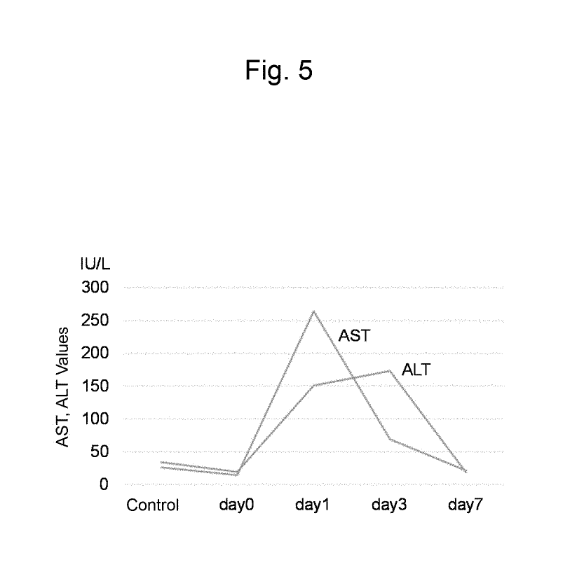

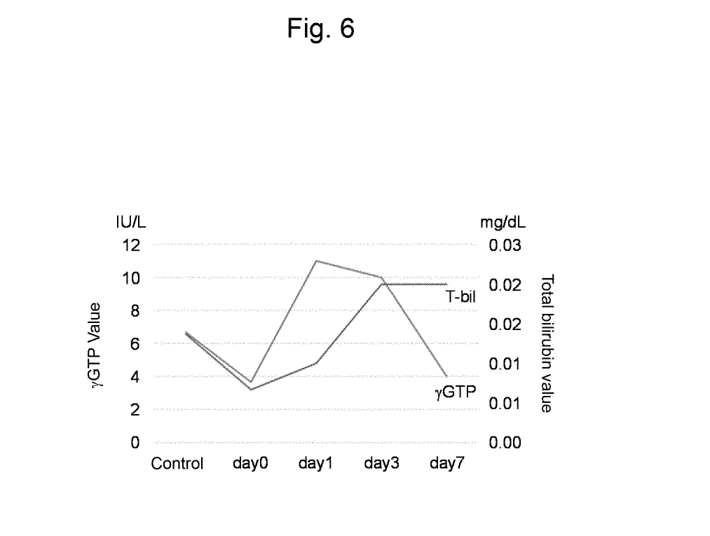

The influence to the liver by the intraportal embolus was also confirmed by the blood biochemical examination. As shown in FIG. 5, with AST peak at Day 1 and ALT peak at Day 3, liver damage having a maximum of almost less than 300 IU/L was detected but on Day 7 the both were alleviated to the same extent as the control. These changes are similarly found in γGTP (FIG. 6), and the total bilirubin, despite a slightly high value on Day 7, did not increase so notable as to suggest the sign of a liver failure. As described above, the preparation for forming emboli according to the present invention was confirmed to be gelled and embolically effective in a blood vessel. Further, the gelation and the embolic effect in a blood vessel was confirmed by the administration of the preparation for forming emboli using the microcatheter of the present invention. Liver damage was found as the adverse effects caused by the intravascular administration of the preparation for forming emboli but it was revealed that each damage was transient and showed the tendency of alleviation on Day 7.

Note that the present invention is not limited to the embodiments described above and encompasses various modifications. For example, a part of the embodiment components may have addition, deletion or substitution from or with other components.

### REFERENCE SIGNS LIST

1 Microcatheter
1a Distal end
1b Proximal end
10 Catheter body
100 Partition walls
101 Inner cavity
102 Outer cavity
103 Mixing space
104 Connecting part
105 Connecting part
L₁, L₂ Liquids

All publications, patents and patent applications cited herein shall be incorporated per se by references in the specification.

1-9. (canceled)

10. A microcatheter having a tubular catheter body that opens at a distal end,
wherein an inner cavity and an outer cavity coaxially partitioned by a partition wall are formed inside the catheter body,
the inner cavity and the outer cavity each opens at a distal end side of the catheter body, and the partition wall is disposed back from the distal end of the catheter body toward a proximal end side, and thereby a mixing space for mixing liquids supplied to the inner cavity and the outer cavity respectively is formed between the opening of the inner cavity and the distal end of the catheter body,
wherein the inner cavity is for supplying either one of a first solution or a second solution, the first solution comprising a phenolic hydroxyl group-modified polymer represented by the following formula (1): 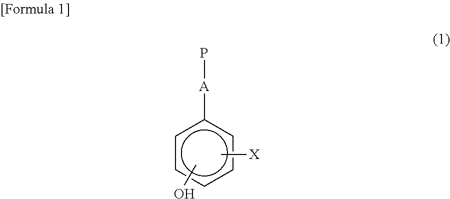
wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group and at least one selected from a peroxidase, a laccase, a tyrosinase, a catalase and an iron porphyrin complex, and
the second solution comprising hydrogen peroxide, and
the outer cavity is for supplying the other.

11. (canceled)

12. The microcatheter according to claim 10, wherein the biocompatible polymer is a gelatin, a pectin, an albumin, a hyaluronic acid, an amylopectin, a chitosan, an alginic acid, a modified polyvinyl alcohol having a carboxyl group, a carboxymethyl cellulose or a collagen.

13. The microcatheter according to claim 10, wherein the phenolic hydroxyl group-modified polymer represented by the formula (1) is a sugar beet-derived pectin having a ferulic acid group.

14. A preparation for forming emboli, being a liquid before injection into a living body but gelled by a crosslinking reaction after the injection to the living body, the preparation comprising:
one to three solutions comprising a phenolic hydroxyl group-modified polymer represented by the following formula (1): 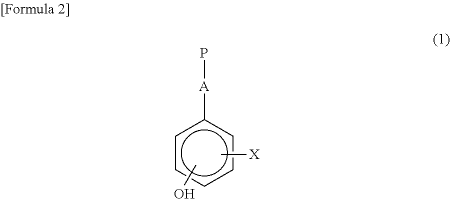
wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group,
at least one selected from a peroxidase, a laccase, a tyrosinase, a catalase and an iron porphyrin complex, and
hydrogen peroxide or at least one selected from a glucose oxidase, a choline oxidase, an amino acid oxidase, an alcohol oxidase, a pyruvate oxidase and a cholesterol oxidase in the same or different solutions,
wherein hydrogen peroxide is not comprised in the same solution as that comprising the phenolic hydroxyl group-modified polymer represented by the following formula (1) and the at least one selected from a peroxidase, a laccase, a tyrosinase, a catalase and an iron porphyrin complex.

15. The preparation according to claim 14, comprising:
a solution comprising a phenolic hydroxyl group-modified polymer represented by the following formula (1): 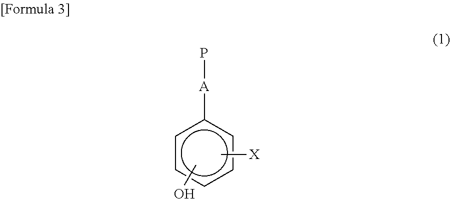
wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group,
a solution comprising at least one selected from a peroxidase, a laccase, a tyrosinase, a catalase and an iron porphyrin complex, and
a solution comprising hydrogen peroxide.

16. The preparation according to claim 14, comprising:
a first solution comprising a phenolic hydroxyl group-modified polymer represented by the following formula (1): 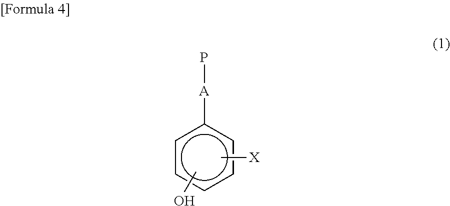
wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group and at least one selected from a peroxidase, a laccase, a tyrosinase, a catalase and an iron porphyrin complex, and
a second solution comprising hydrogen peroxide.

17. The preparation according to claim 14, comprising:
a solution comprising a phenolic hydroxyl group-modified polymer represented by the following formula (1): 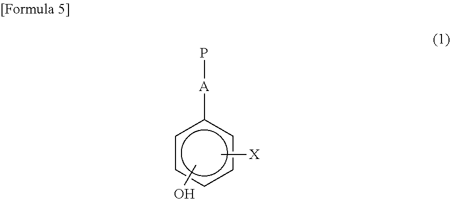
wherein P is a biocompatible polymer, A is a single bond or an —OCO—C₂-C₄-alkenylene group, a —CONH—C₁-C₄-alkylene group or an —HNCO—C₁-C₄-alkylene group, and X is hydrogen or a C₁-C₃-alkoxy group at least one selected from a peroxidase, a laccase, a tyrosinase, a catalase and an iron porphyrin complex and at least one selected from a glucose oxidase, a choline oxidase, an amino acid oxidase, an alcohol oxidase, a pyruvate oxidase and a cholesterol oxidase.

18. The preparation for forming emboli according to claim 14, wherein the biocompatible polymer is a gelatin, a pectin, an albumin, a hyaluronic acid, an amylopectin, a chitosan, an alginic acid, a modified polyvinyl alcohol having a carboxyl group, a carboxymethyl cellulose or a collagen.

19. The preparation for forming emboli according to claim 14, wherein the phenolic hydroxyl group-modified polymer represented by the formula (1) is a sugar beet-derived pectin having a ferulic acid group.

20. The preparation for forming emboli according to claim 14, comprising, in addition to the phenolic hydroxyl group-modified polymer represented by the formula (1), a different phenolic hydroxyl group-modified polymer represented by the formula (1).

21. The preparation for forming emboli according to claim 14, comprising, in addition to the phenolic hydroxyl group-modified polymer represented by the formula (1), aniline or a derivative thereof.

22. The preparation for forming emboli according to claim 14, further comprising an agent.

23. The preparation for forming emboli according to claim 22, wherein the agent is an anticancer agent.

24. A method for forming emboli comprising:
injecting the preparation according to claim 14 into a living body, and
gelatinize the preparation by a crosslinking reaction to form emboli.
